(19) United States
(12) Patent Application Publication
(10) Pub. No.: US 2025/0177740 A1
(43) Pub. Date: Jun. 5, 2025

(54) METHOD OF REDUCING STIMULATION ARTIFACT INDUCED BY AN ELECTRICAL STIMULATOR IN NEUROPHYSIOLOGY AND ELECTRICAL STIMULATION DEVICE FOR PERFORMING THIS METHOD

(71) Applicant: DEYMED DIAGNOSTIC S.R.O., Hronov (CZ)

(72) Inventor: Miroslav MORAVEC, Hronov (CZ)

(21) Appl. No.: 18/844,572

(22) PCT Filed: Mar. 8, 2023
(86) PCT No.: PCT/CZ2023/000009

§ 371 Date: Sep. 6, 2024

(30) Foreign Application Priority Data
Mar. 9, 2022 (CZ) PV 2022-110

(51) Int. Cl. A61N 1/08

(52) U.S. Cl. CPC A61N 1/08

(57) ABSTRACT

A method of reducing stimulation artifact when performing electrical stimulation, in which a stimulation pulse is generated by an electrical stimulator connected to a stimulation electrode. A closed equipotential surface is created around the electrical stimulator by complete electrical shielding of the electrical stimulator from the rest of the electrical stimulator and from the surroundings, a low capacitive coupling is ensured between the electrical stimulator itself and the rest of the device structure, the shielding of the stimulation electrode is connected to the closed equipotential surface of the electrical stimulator and to the closed equipotential surface electrical stimulator and/or a collection electrode designed to be placed on the patient is connected to the shielding of the stimulation electrode. The stimulation artifact reducing electrical stimulation device comprises an electrical stimulator (1) designed to connect to a stimulation electrode. The electrical stimulator (1) is provided with a shield (3) with an electrically shielding surface for shielding the electrical stimulator by creating an equipotential conductive surface surrounding this electrical stimulator (1), wherein the shielding is provided with a connection connector for connection to the shielding of the stimulation electrode or is directly connected to this shielding, and that the shielding (3) of the electrical stimulator (1) and/or to the shielding of the stimulating electrode is designed to be connected to the collecting electrode (6) or is connected to the collecting electrode (6), and that at least between the electrical stimulator (1) and the remaining parts of the device create a low capacitive coupling.

### FIELD OF THE INVENTION

The invention relates to a method of reducing stimulation artifact during electrical stimulation with an electrical stimulator in neurophysiology and an electrical stimulation device for performing this method in neurophysiological examinations, wherein the device contains an electrical stimulator and exhibits reduced stimulation artifact.

### STATE OF THE ART

Stimulation artifact is a disturbing phenomenon in the electrophysiological signal that appears in neurophysiological measurements almost everywhere where electrical stimulation is used. It is caused by the penetration of disturbing voltages and currents escaped from the electrical stimulator, the stimulation electrode and from the stimulated parts of the patient into the tissues from which the electrophysiological responses are measured, respectively. penetrating directly into the measuring circuits.

In neurophysiology, various methods of electrophysiological examination are used, which use electrical stimulators to stimulate the organism and the registration part to determine the reaction of the organism to this electrical stimulation, while the following fields are mainly involved: electromyography (EMG), electrocorticography (ECoG), transcranial electrical stimulation (TES), intraoperative monitoring (IOM), deep brain stimulation with feedback (close loop VNS) and others.

The most common use of electrical stimulators is currently in electromyography, where devices allowing this type of neurophysiological examination currently represent the majority of electrical stimulator applications. In order to induce an electrophysiological response, the vast majority of current types are chosen, in which a constant size of the stimulation current is maintained during the entire course of stimulation, regardless of the output load impedance. In the vast majority, such stimulators are used for transcutaneous (through intact skin) stimulation, but under restrictive conditions they can also be used together with needle electrodes for direct stimulation of nerves or muscles.

Devices used in electromyography, hereinafter referred to as EMG devices, but also devices used in other methods of electrophysiological examinations mentioned above, contain an electrical stimulator performing electrical stimulation and a registration part performing an evaluation of the organism's response to this electrical stimulation. An electrical stimulator generating an electrical stimulation signal is used, for example, in EMG to stimulate nerve tissue, or directly to the muscle. The registration part then monitors the organism's physiological reaction to this stimulus and is made up mainly of a signal amplifier, with registration electrodes connected to this signal amplifier. In these methods, electrical stimulators generate short and relatively intense electrical current stimulation pulses of selectable polarity, while the electrophysiological response of the nerve is registered, or muscle, or inducing mechanical movement of the muscle, which is measured in a different way. The width of the electrical stimulation pulses ranges from approx. 20 μs to approx. 1 ms. The repetition of these pulses then reaches a frequency of up to 300 Hz, but in this case only a short sequence of individual pulses is used, approximately up to ten pulses. The dependence of the intensity of excitation of an individual neuron is not linearly dependent on the intensity of stimulation, but it has a threshold character. In order to induce full irritation, the threshold polarization of its cell membrane, which is on the order of tens of millivolts, needs to be approximately equalized. When the stimulation impulse has a lower intensity, irritation would not occur. After the creation of the stimulation signal, the reaction of the organism to the irritation is recorded in the registration part. This reaction has a character of an electrophysiological response of neurons that are connected to the stimulated neurons, or of directly stimulated neurons, or also the response of the respective muscle, which is innervated by excitation leading neurons.

Electric current stimulators used in the above-mentioned diagnostic methods of electrophysiological examinations, especially in electromyography, mainly contain:
high-voltage amplifiers with current output ensuring a constant size of the stimulation current regardless of the load impedance,
energy storage, most often capacitors, providing enough energy and power for short intense stimulation, so that a powerful source is not necessary, whereas such powerful source would unnecessarily enlarge the structure of the stimulator and make it more expensive, while the limited power output of the rechargeable source also contributes to increasing the safety of the device,
an amplifier with a current output with a maximum amplitude of the output current from approx. ±15 mA to approx. ±1 A, typically 100 mA, while the magnitude of the voltage ranges from approx. 30 V to approx. 1000 V depending on the size of the load impedance, the voltage typically having a value of around 400 V;
a voltage limitation detection circuit of a current output in case of a load having high impedance, when the further increase in voltage is no longer sufficient to supply the set level of the stimulation current.

These electrical stimulators are connected to control circuits controlling a start of the electrical stimulation and the also controlling the registration part for monitoring the electrophysiological response to the electrical stimulation.

The output of the electrical stimulator is connected to the stimulation electrode. A pair of stimulation poles, anode and cathode, which are connected to the patient, is called a stimulation electrode for the purposes of this invention. This pair of stimulation poles is most often fixed together, thanks to which a defined distance between the anode and the cathode is ensured. It is known that the anode-cathode spacing of the stimulation electrode is simply a compromise between accuracy and depth of the stimulation. With a large anode-cathode spacing, it is possible that the stimulation will be carried out even in distant structures from the cathode, on the contrary, with a small anode-cathode spacing, stimulation of the nerve tissue may not occur at a necessary depth, because the electric current induced by the stimulating electrode would not reliably reach the necessary intensity. The spacing between the cathode and the anode of the stimulation electrode in human medicine usually ranges from 20 mm to 40 mm, for children it can even be as little as 10 mm, e.g. in electromyography, electrodes with a spacing between the centers of the cathode and the anode of 23 mm are most often used.

The recording electrodes are connected to the recording part, so that they are essentially part of it, and serve to sense the response to the electrical impulse at the desired location of the patient's body. The registration electrodes are placed on the patient at the required distance from the stimulation electrode. For example, in electromyography, the electrical stimulation described above is most often used in such a way that the stimulation electrode is placed, for example, on a finger of the hand, while the recording electrodes are placed, for example, on the wrist, elbow, shoulder, spine or on the scalp, the stimulation electrode can also be placed, for example, on an ankle of the leg, while the registration electrodes are placed, for example, under the knee, on the spine or on the scalp.

As already mentioned, during electrical stimulation, so-called stimulation artifacts arise, which make evaluation of the measured electrophysiological response difficult and negatively affect it, e.g. by making the estimation of the beginning of the response difficult, for example. The amplitude of the initial stimulation artifact is not so decisive for the quality of the evaluation of the electrophysiological response to the electrical stimulus, but especially the amplitude and duration of the so-called decay artifact, which follows it, intervening in time up to the response itself being measured.

Different methods are known for removing stimulation artefacts caused by repetitive electrical stimulation from a native electrophysiological signal, these methods being based on finding a regularly repeating pattern of the artefact, but from the principle of their function they also remove their own evoked response, being used for applications that are intended for evaluation of native activity without analyzing one's own evoked response. Furthermore, methods based on the modeling of the artifact during subthreshold stimulation using the strong nonlinearity of the electrophysiological response to the stimulus intensity are also known. Therefore, they mainly analyze the signal during one or several subthreshold stimulations of different intensity, when the own electrophysiological response is not present or is present only in a very small amplitude. Subsequently, a model of the stimulation artifact is created, which is subsequently subtracted from the evoked response. Furthermore, there are known methods using the modeling of an indirect stimulation artifact based on its approximately known parameters, and many others. However, none of the known software methods addresses a reliable and sufficient removal of stimulation artifacts.

### SUBJECT OF THE INVENTION

The above-mentioned complications caused by stimulation artifacts are eliminated or at least substantially reduced by a method of reducing the stimulation artifact caused by the electrical stimulator in neurophysiology and the electrical stimulation device according to the present invention. For the purposes of this invention, by the term electrical stimulation a creation of an electrical impulse by an electrical stimulator of this electrical stimulation device is meant, its application to an organism's body, or on a patient, and subsequent measurement of the reaction to this stimulation impulse by registration electrodes placed at the appropriate place on the organism's body, or on the patient, the registration electrodes being connected to the registration part of the device for electrical stimulation.

According to one aspect of the present invention, a method of reducing stimulation artifact during electrical stimulation is disclosed, in which a stimulation pulse is generated by an electrical stimulator connected to a stimulation electrode in a device for performing the electrical stimulation, the stimulation pulse being subsequently measured by a recording electrode connected to a recording part of the device for performing the electrical stimulation, whereby:
a) a closed equipotential surface around the electrical stimulator is created, wherein this closed equipotential surface is created by completely electrically shielding the electrical stimulator by means of an electrical shielding;
b) a low capacitive coupling is provided between the electrical stimulator and the rest of the parts of the device for performing electrical stimulation,
c) a shielded stimulation electrode is connected to the closed equipotential surface of the electrical stimulator, while its shielding is electrically connected to the electrical shielding of the electrical stimulator;
d) the closed equipotential surface of the electrical stimulator and/or the electrical shielding of the stimulation electrode is electrically connected to a collection electrode designed to be placed on the patient, the collection electrode being placed between the stimulation electrode and the recording electrode at a distance corresponding to at most % of the distance between the recording electrode and the stimulation electrode.

For the purposes of this invention, by a low capacitive coupling is called a coupling having an order of pF units. Said low capacitive coupling between the electrical stimulator and the rest of the electrical stimulation device is important in order to create an isolated peninsula, said isolated peninsula will be described below. The term “the rest of the parts of the device for carrying out electrical stimulation” Incorporates in particular the registration part with the registration electrode, preferably the control part as well, the power supply part, the communication part and any other parts of this device.

Low capacitive coupling is mainly achieved by using the battery power supply of the electric stimulator or by using an electric stimulator power source with a low coupling capacity against the surroundings.

According to a particularly advantageous embodiment of the method according to the present invention, formation of a current loop in the patient, bypassing the connection point of the collection electrode during the stimulation is avoided. If such a current loop were to be created, for example by touching the end of the stimulated limb with the patient's body behind the point of connection of the collecting electrode on the patient, the equalizing currents created could affect the resulting signal sensed by the recording electrode connected to the recording part. Likewise, it is particularly desirable to avoid creating a current loop when implanting the entire pacemaker into the patient.

A closed equipotential surface around the electrical stimulator is created by performing electrical shielding of this electrical stimulator. The term electrical shielding of the electrical stimulator means that the electrical stimulator is electrically shielded from the rest of the device for the electrical stimulation, especially from its registration part with registration electrodes. This electrical shielding of the electrical stimulator can be done in many ways, e.g. by enclosing the electrical stimulator in a cover made of electrically conductive material/e.g. aluminum, sheet metal, etc.), by enclosing the electric stimulator in a cover made of electrically non-conductive material provided on the outside and/or inside with electrically conductive material, for example by complete foiling with an electrically conductive foil or by spraying an electrically conductive paint, etc. The resistance of this electrically conductive material should be at most about 100 Ohm per square, more preferably 10 Ohm per square and most preferably 1 Ohm per square. To ensure a closed equipotential surface, all connections of the electrical stimulator, e.g. the stimulation electrode connection, are made isolated from the electrically conductive surface, if connection with this surface is not required, like in case of the collection electrode or in case of the shielding of the stimulation electrode. Due to the requirement to create a low capacitive coupling of the electrical stimulator to the rest of the electrical stimulation equipment, the power supply of the electrical stimulator is particularly advantageously carried out by a battery supply or by a power supply with a very low coupling electrical capacitance between the electrical stimulator and the power circuit, the coupling capacitance being at most units of pF. According to a particularly advantageous implementation of the device for performing electrical stimulation according to the invention, this requirement for a low coupling capacity also applies to the connection of the electrical stimulator with the control element and all possible communication elements. For the connection of the stimulation electrode, it is sufficient to use a shielded conductor with a standard shielding quality, possibly using a shielding spray or an electroconductive foil, with the above-mentioned electrical resistance. A common electrode with one pole, identical in type to the grounding electrode, is preferably used as the collecting electrode. It is more advantageous to use a collection electrode with a larger active surface, due to the achievement of a lower transient impedance and thus a higher suppression of the stimulation artifact.

According to the second aspect of the present invention, a device for electrical stimulation reducing stimulation artifact is provided, comprising in particular an electrical stimulator and a registration part, where this electrical stimulator is provided with an electrically shielded stimulation electrode for conducting electrical stimulation, and this electrical stimulator is further provided with electrical shielding for its complete electrical shielding and has a connected, electrical shielding of the stimulation electrode with the electrical shielding of the electrical stimulator, or is designed for their connection, while the electric stimulator is further provided with a collecting electrode, wherein this collecting electrode is connected, or designed to be connected to the electrical shielding of the electrical stimulator and/or the electrical shielding of the stimulation electrode in order to minimize the electrical capacitance between the electrical stimulator and the patient.

According to a particularly advantageous implementation of the device according to the invention, at least the electric stimulator, possibly also the registration part, has the smallest possible dimensions, thus reducing their mutual electric capacity and the capacity of the electric stimulator with respect to a room in which the device is located. There is a rule, that objects having a cuboid-like shape with a small aspect ratio of their walls, disclose their electrical capacity in pF, with respect to the room, in a value of approximately half the size of the largest dimension of said object in cm.

The method and device according to the present invention are based on an idea of removing influence of the relatively high in parallel connected electrical capacitance of the stimulation electrode cable and achieving a low electrical capacitance of the electrical stimulator against the patient, wherein thanks to the features of the invention an “insulated peninsula” is essentially created around the electrical stimulator, the stimulating electrode and the stimulated part the body of the patient by connecting the electrical shield of the electrical stimulator with the collection electrode, said “insulated peninsula” prevents stimulation artifact from entering the registration site, as will be described below. By creating this “isolated peninsula” there is reduced capacitive coupling between the stimulation electrode of the electrical stimulator and the non-stimulated biological tissues on which the registration electrodes are fixed, through which the necessary measurement is performed. This “isolated peninsula” can be created, for example, on a stimulated finger or the entire limb. According to this invention, either by applying the method according to the invention or thanks to the features of the device for implementing this method, the majority of the electric charge generated during electrical stimulation is led back to the collecting electrode, thereby preventing the further spread of the majority of the electric charge to the registration site. A certain amount of the charge spreads further, but it has a significantly lower size and no longer significantly affects the measured response. This invention can also be viewed in such a way that thanks to it a T cell is created dividing the coupling capacitance into two separate capacities and grounding its dividing line, thereby interrupting the transmission using the coupling capacitance.

To ensure the reliable removal of unwanted artifacts, the contact of an end of the limb with the patient's body should be avoided, as an equalization current would begin to flow through the limb, which would negatively affect the correct function of the proposed solution. If this rule is known, it is easy in practice to ensure its observance.

### DESCRIPTION OF DRAWINGS

The invention will be more easily and clearly understood from the following examples of implementation and from the accompanying drawings, in which:

FIG. 1 shows a comparison of the stimulation artifact in the device according to the state of the art with the stimulation artifact in the device according to the subject of the invention,

FIG. 2a shows a simplified device connection model according to the state of the art,

FIG. 2b is a schematic drawing showing the propagation of the direct stimulation artifact induced by the electrical stimulator,

FIG. 3a shows a schematic illustration of the connection of the device according to the invention to the patient,

FIG. 3b shows a simplified device connection model according to the state of the art,

FIG. 4a schematically shows the connection of the electrical stimulator of the device according to the invention to the patient's finger,

FIG. 4b shows schematically the connection of the electrical stimulator of the device according to the invention to the limb of the patient,

FIG. 5a shows the dependence of the shape of the stimulation artifact on the distance from the stimulation site in devices according to the state of the art,

FIG. 5b shows the dependence of the shape of the stimulation artifact on the distance from the stimulation site in the device according to the invention,

FIG. 6 shows the result of the examination of somatosensory evoked potentials on the device according to the invention,

FIG. 7a shows a comparison of the results of the examination of the sensory nerve on the device according to the invention with the results of the examination on the device according to the state of the art,

FIG. 7b corresponds to the image from FIG. 7a with reduced sensitivity,

FIGS. 8a and 8b represent the dependence of the size of the stimulation artifact for several distances of the connection point of the collection electrode from the stimulation site,

FIG. 9 shows the device according to the invention connected to the upper limb of the patient including the registration part.

### EMBODIMENTS OF THE INVENTION

For a better understanding of the electrical stimulator according to the present invention, embodiments of its advantageous implementation will now be described. Even specific embodiments will be used to describe the invention, with a reference to certain drawings, the invention is not limited to the described embodiments and is limited only by the claims. The attached drawings are schematic only and are in no way intended to limit the invention to the embodiments shown. In the drawings, the size of some elements may be exaggerated for illustrative purposes and these may not be drawn to scale. The dimensions and their relative proportions may not correspond to the actual dimensions. Furthermore, although some embodiments of the invention described herein include only some elements, not the other elements, while these other elements are included in other embodiments, combinations of elements from different embodiments are possible to fall within the scope of the invention and form embodiments other than those described herein, which will be fully understandable to men skilled in the art. For example, the embodiments described in the exemplary embodiments may be used in any suitable combination. Similar parts of the device for electrical stimulation are marked with the same reference numbers in the individual drawings. Furthermore, it should be noted that the features of the device in the embodiment of the invention in the individual images can be combined in any way, if it is possible.

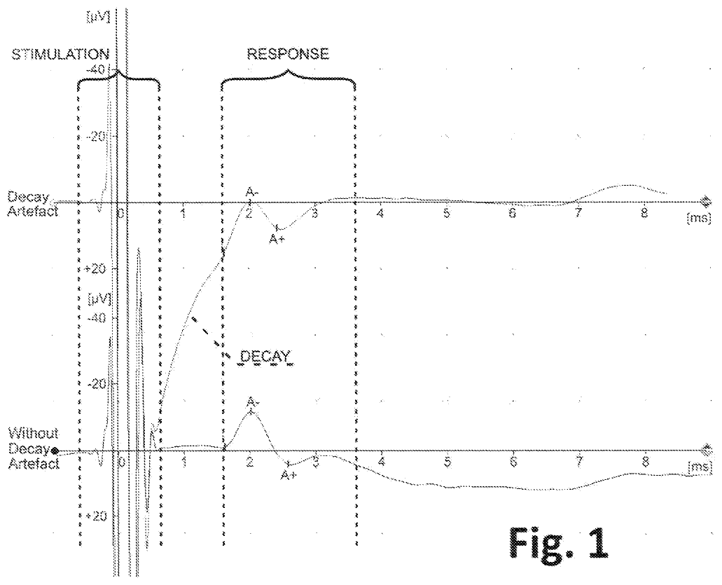

FIG. 1 shows a comparison of signal curves measured by the recording electrodes during the stimulation performed by a device according to the state of the art and by a device according to the subject of the invention with a current of 6.5 mA and a duration of 200 μs, which was large enough to ensure the completeness of the response. As can be seen from FIG. 1, in the initial phase up to about 0.2 ms, the curve is influenced by the stimulation impulse itself, which is followed by a decay artifact in the stimulator according to the state of the art, followed in about 1.5 ms by the organism's response to the stimulation impulse, which starts at the point marked with an index/marker/I. An amplitude of the response is then marked with indices/markers/A+, A−.

Subsequently, a method according to the invention will be described as well as a device for performing said method. FIG. 1 shows neurograms of a sensitive nerve scanned by recording electrodes in a device according to the state of the art and in a device on which the artefact reduction method according to the invention was applied. As can be seen from FIG. 1, in the initial phase up to about 0.2 ms, the curve is influenced by the stimulation impulse itself, which is followed by a decay artifact in the state-of-the-art stimulator, followed in about 1.5 ms by the response of the nerve to the performed stimulation, where this response begins at the point indicated by the index/marker/I. The amplitude range of the response is then indicated by the indices/markers/A+, A−. As can be seen from the upper graph, it is difficult for the prior art device to find out where the intrinsic response begins, because up to the point I, which marks the beginning of the response, the curve rises due to the artifact and the point I is difficult to identify on it. In the lower curve, on the other hand, there is no artifact and the point I indicating the beginning of the answer is easy to find. It is clear from FIG. 1 that the stimulation artifact consists of two parts, the initial stimulation artifact, labeled as “stimulation,” and the subsequent decay artifact, labeled as decay. The initial stimulation artifact is not as serious for response measurement as the decay artifact, because it is clearly limited in time and lasts very briefly, basically only for the duration of the stimulation, while the decay artifact decays for a very long time, on the order of tens of ms, and as shown in FIG. 1 obvious, it is difficult to distinguish from it when the response of the nerve begins, compared to the well-defined beginning of the response of the device according to the state of the art “without decay artifact”). The amplitude of the initial stimulation artifact (marked as “STIMULATION” in FIG. 1) is therefore not so decisive for the quality of the evaluation of the electrophysiological response to an electrical stimulus, but rather the amplitude and duration of the so-called decay artifact (marked as “DECAY” in FIG. 1) which follows it and extends in time to the response itself (marked as “RESPONSE”) that is measured.

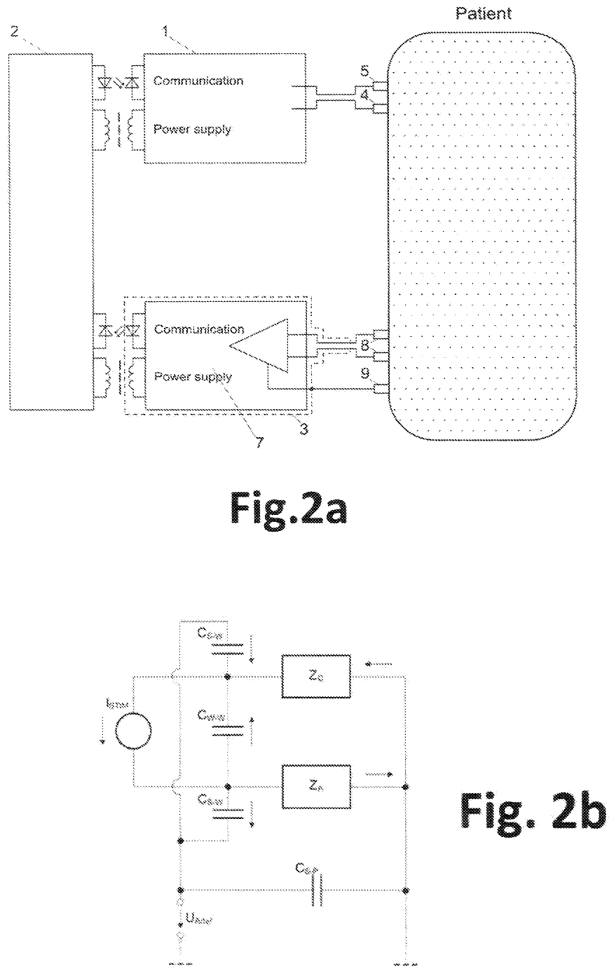

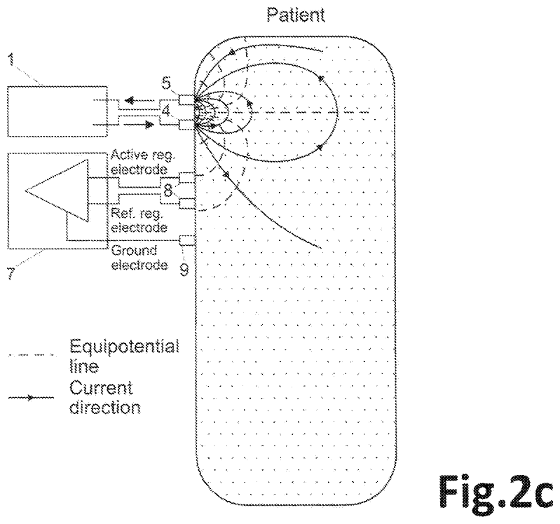

FIG. 2a shows a simplified connection model of a state-of-the-art electrical stimulation device connected to the patient. This device includes an electrical stimulator 1, a control unit 2, a stimulation electrode with anode 3 and cathode 4, a registration part 7 with registration electrodes 8 and a ground electrode 9. The following quantities are further shown in FIG. 2a:

Z_{C} is the transition impedance between the cathode of the stimulation electrode and the patient

Z_{A} is the transition impedance between the anode of the stimulation electrode and the patient

C_{S-W} is the capacity between the shielding of the connection cable of the stimulation electrode and one wire to connect the anode, or cathodes

C_{W-W} is the capacitance between the anode and cathode leads of the stimulation electrode connection cable

C_{S-P} is the capacitance between the electrical stimulator and the patient

C_{S-U} is the capacitance between the electrical stimulator and the control unit

C_{B-U} is the capacitance between the registration part and the control unit

C_{S-B} is the capacitance between the electrical stimulator and the recording part

It is clear to a man skilled in the art that the above-mentioned capacities are actually parasitic capacities.

I_{STIM} is a current flowing through the patient that generates an electrical stimulator

U_{ARTEF} is a voltage on the shielding of the connecting cable of the stimulation electrode measured to the patient.

FIG. 2b is a schematic drawing showing a propagation of the direct stimulation artifact induced by the electrical stimulator on the patient's body, from which it is also evident how it extends into the region of the recording electrodes.

A method of reducing the stimulation artifact when performing electrical stimulation, in which a stimulation pulse is generated by an electrical stimulator connected to a stimulation electrode, was carried out as follows. A closed equipotential surface was created around the electric stimulator by completely electrically shielding the electric stimulator from the rest of the device and from the surroundings by applying an electrically conductive foil to the cover of the electric stimulator from the inside, while this foil had an electrical resistance of 100 Ohm per square. A low capacitive coupling between the electric stimulator itself and the rest of the device structure, including the room, was ensured thanks to the battery power supply of the electric stimulator and an optical connection of an output of the control unit with an input of the electric stimulator. A shielding of the stimulation electrode was connected to the closed equipotential surface of the electric stimulator, created by said shielding, and a collection electrode was connected to this shielding. This collection electrode was placed on the patient at a distance of 15 cm from the stimulation electrode, wherein the recording electrode was placed at a distance of 40 cm from the stimulation electrode.

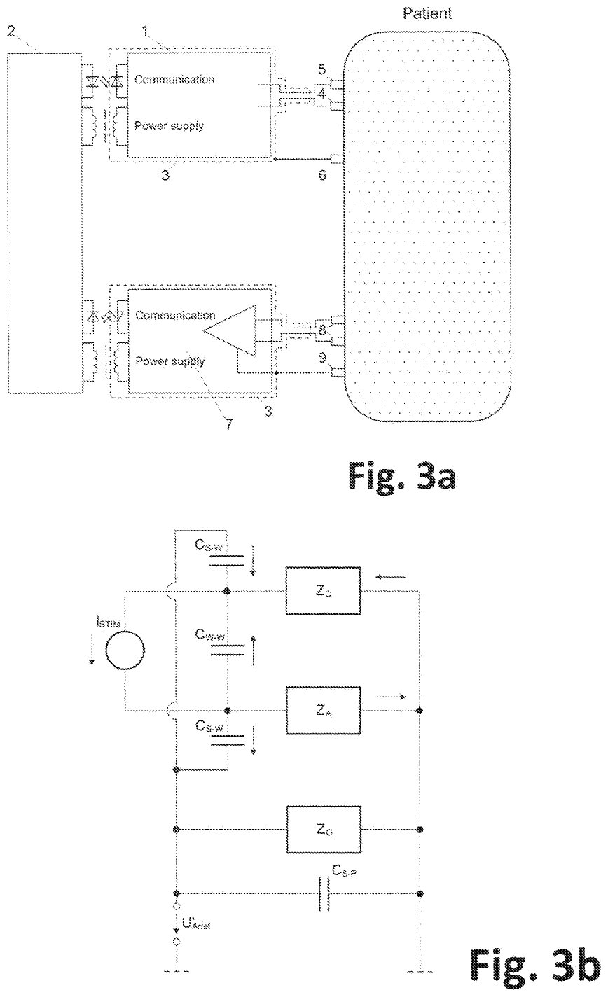

The device according to the invention is then schematically depicted in FIG. 3a, in which the electric stimulator 1 is capacitively separated from the control unit 2 and from the registration part 7, designated here as the amplifier box. Electric stimulator 1 is equipped with shielding 3 and is connected to a stimulating electrode with anode 4 and cathode 5. It is also equipped with collecting electrode 6. As electric stimulator 1, a single-channel electric current stimulator of the TruTrace electromyograph manufactured by Deymed, placed in a plastic cover, was used. The registration part 7 was formed by one channel of a standard four-channel box of EMG amplifiers of the same electromyograph. The low capacitive coupling was ensured by the fact that the electrical stimulator 1 and the box of EMG amplifiers of the registration part 7 were supplied via isolation transformers with a very low capacitive coupling between the windings, being in the order of picofarad units. The electrical shielding 3 of the electrical stimulator 1 was made by an internal electroconductive coating of the plastic cover of the electrical stimulator 1, said coating having an electrical resistance in the order of Ohm units per square. This electrical shielding 3 was externally derived and formed to enable the connection of the collecting electrode 6. For the electrical stimulation, a shielded bipolar stimulating electrode having an anode 4 and a cathode 5, with a length of its connection cable of 2 m and a spacing between the centers of the anode 4 and the cathode 5 was chosen, which were in a form of wetted felt with a diameter of 23 mm. The shielding of this stimulation electrode was electrically connected to the electrical shielding 3 of the electrical stimulator 1 and connected to the patient in the immediate vicinity of the stimulation site by means of a collection electrode 6, with the collection electrode 6 placed between the cathode 5 and the anode 4. For the collection electrode 6, a standard tape ground was used electrode with a connecting cable length of 2 m and a tape width of approx. 1.5 cm. A shielded pair of adhesive pre-gelled recording electrodes widely used for recording electrophysiological responses was chosen for recording, connected by a 2 m shielded cable to one channel in the recording part 7 of the device. All consumable supplies represent probably the most commonly used standard used routinely in most well-equipped clinical European workplaces. The dimensions of the electric stimulator 1 and the amplifier box forming the registration part 7 are approximately 14 cm×9 cm×4 cm.

The current flowing through the collection electrode 6 itself, which conducts the residual charge from the stimulation electrode, could theoretically affect the stimulation parameters or cause stimulation at an unwanted place where the collection electrode 6 is connected, therefore its size is calculated. Since the current flowing through the collection electrode 6 has a pulse character and flows only at the beginning and end of the stimulation pulse, the electric charge flowing through this collection electrode 6 is considered to determine the electrophysiological effect, as it is responsible for the resulting electrophysiological effect.

For the stimulation itself, the highest possible stimulation current of 100 mA is considered and the typically used stimulation width is 200 pts, the charge causing the stimulation is then 20 μC. Even with such strong stimulation, the current flowing through the collecting electrode should not be able to significantly influence the temporal or spatial parameters of the stimulation, which can also be verified by subsequent calculation.

The shielded lead cable used to connect the stimulation electrode had an electrical capacity of the individual conductor against the shield of approx. 100 pF/m, the capacitance of the connected conductors against the shield was approx. 200 pF/m, which leads to a final electrical capacity of 400 pF with a cable length of 2 m. The capacitor created in this way is charged to a voltage of half the saturation voltage under that condition. Since the saturation voltage reaches up to 400 V, this means that up to 400 pF×200 V=80 nC of charge must be drained to avoid occurring an artifact affecting the registration. The discharge of the charge was carried out by the collecting electrode 6, which led this charge back to the electrical shield 3 of the electrical stimulator 1, so it was not able to affect the registration site.

A charge of 80 nC then corresponds to a stimulation current of 0.4 mA flowing for 200 μs, which is 0.4% of the stimulation intensity. This means that the ratio of the charge causing the stimulation, which in this case is 20 μC, and the 80 nC charge conducted by the collection electrode is approximately 1:250. This value is calculated considering the worst operating conditions, i.e. connecting the anode 4 with a zero transition impedance and connecting the cathode 5 with an impedance just below the voltage saturation of the current output of the electrical stimulator 1, or exactly the opposite, i.e. an ideally connected cathode 5 and a very poorly connected anode 4. Such an unfavorable combination of conditions does not occur in practice, mainly because it is practically unattainable to perfectly connect the anode or cathode to the patient.

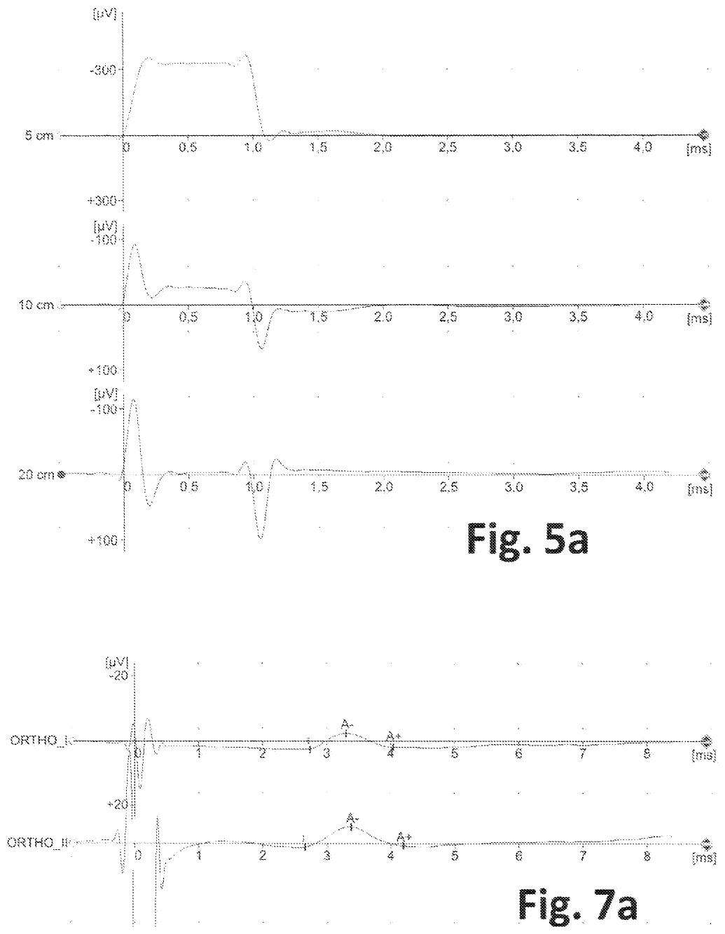

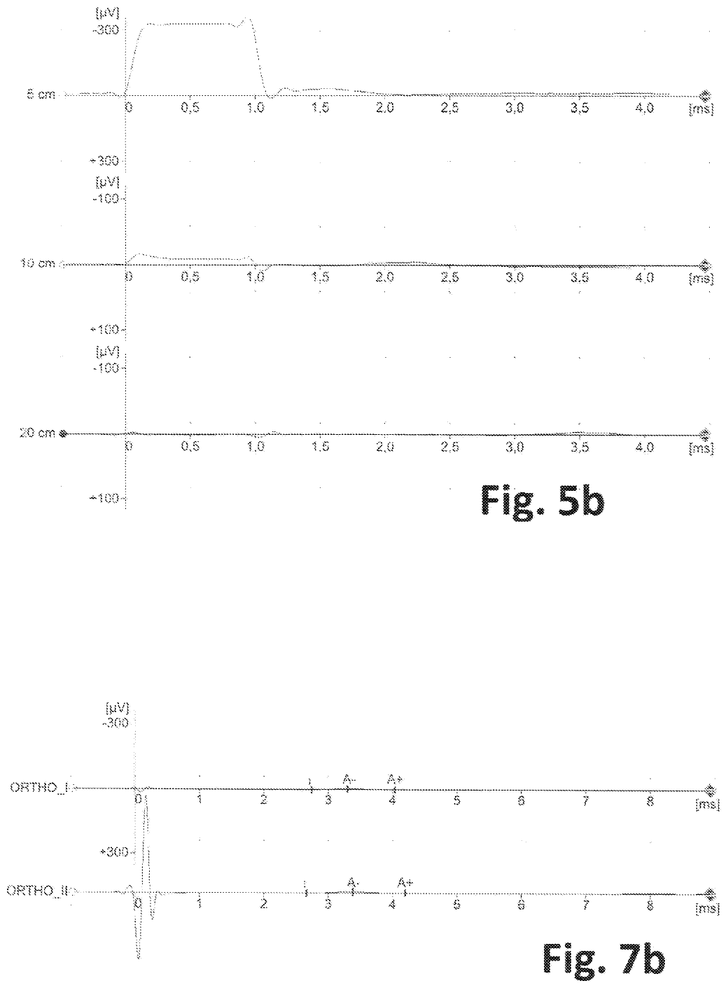

Charge transfers leading the current back to the electrical shield 3 of the electrical stimulator 1 flow through the collecting electrode 6 twice in close succession, the first at the beginning of the pulse, the second at its end, always with opposite polarity, see e.g. the bottom two graphs in FIG. 5a. However, their resulting physiological effect does not need to be multiplied by two, it corresponds approximately to a single pulse of one polarity only. This fact is verified experimentally using the identical physiological effect of an electrical biphasic impulse with twice the duration compared to the effects of an impulse of the same intensity and of one polarity.

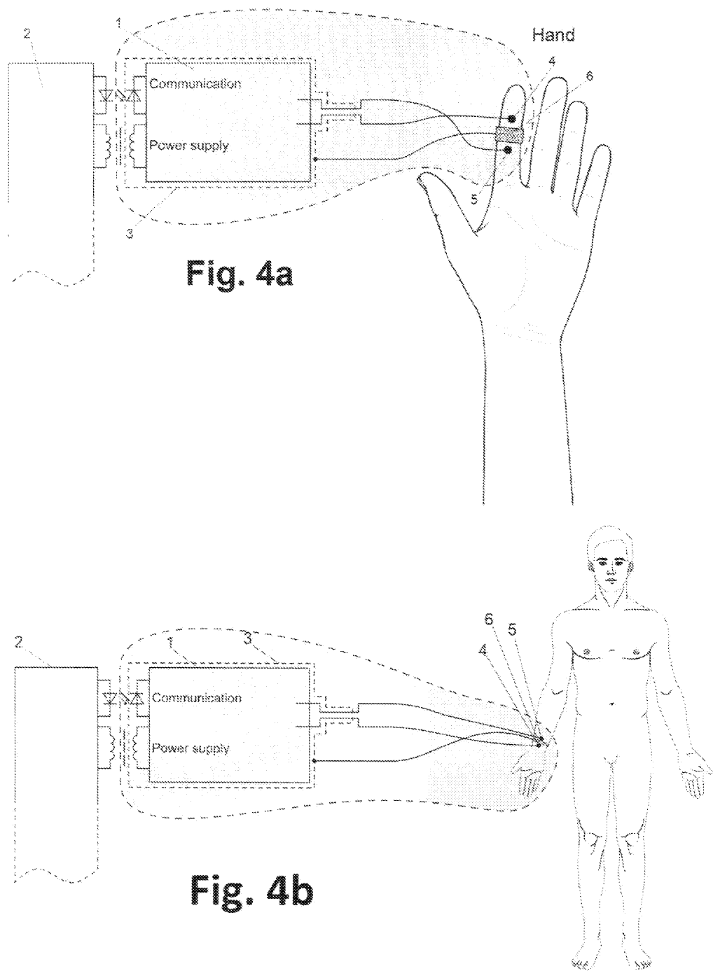

In FIGS. 4a and 4b, the contour of the isolated peninsula 10 is marked with dashed lines, which is formed essentially from the place on the patient's body where the collection electrode 6 is connected to the electrical stimulator, and which is clearly separated from the place of connection of the registration electrodes. In this case, an isolated peninsula 10 was created on the patient's body extending essentially from the point of connection of the collecting electrode 6 to the fingertips of the given limb. The effect of the isolated peninsula 10, created when applying the method according to the present invention, or the device according to the invention, is particularly applicable in cases with the connection point of the registration electrodes being from the connection point of the stimulation electrode at a distance of more than approx. six times the distance between the anode and the cathode of the stimulation electrode. Up to approximately this distance, the direct galvanic transmission of the stimulation impulse through the stimulated biological tissue prevails, and the effect of the collecting electrode according to the invention does not significantly contribute to the reduction of the stimulation artifact, as is also clear from FIG. 5a. FIG. 5a shows the shape of the stimulation artifact in the state-of-the-art device, which is measured by recording electrodes placed at a distance of 5 cm, 10 cm and 20 cm from the stimulation site. It is obvious that up to a distance of approx. 20 cm, the effect of direct galvanic propagation of the stimulation impulse strongly prevails. FIG. 5 b shows the dependence of the shape of the stimulation artifact in the device according to the invention on the distance between the connection point of the registration electrodes and the connection point of the stimulation electrode. It is clear from it that the artefact removal method and the device according to the invention are particularly useful if the registration electrodes are sufficiently far from the connection point of the stimulation electrode, so that only the indirectly propagated stimulation artefact is removed. It is important to note that the reliable connection distance of the registration electrode of 20 cm is valid only for this particular case, which serves only for information on the use of the method and device according to the invention. The indicated reliable distance between the connection point of the registration electrodes and the connection point of the stimulation electrode depends on several factors, in particular on the size of the stimulated object and the distance between the anode and the cathode of the stimulation electrode. It is therefore appropriate to determine a reliable distance for each examination method in which electrical stimulation is used and for each stimulation electrode used.

By conducting most of the electric current, or of the electrical charge, which was created during electrical stimulation, back to the collection electrode 6, which is particularly advantageously located near the stimulation site, the method and device according to the invention largely prevents its spread to the registration site.

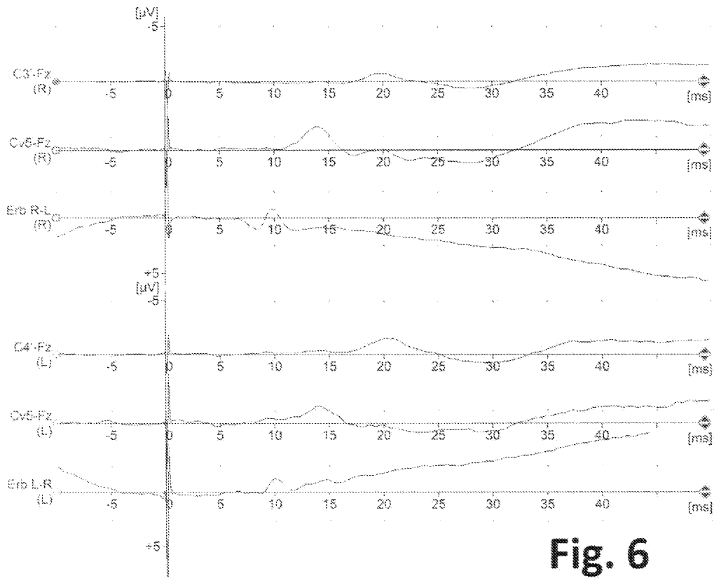

Comparison of examination results using the method according to the invention, or using the device according to the invention, with the results on the prior art device are shown in FIGS. 7a and 7b, both results are measured using the orthodromic stimulation technique. The curves marked ORTHO I in FIGS. 7a and 7b show the result of the measurement with the device according to the invention, and the curves ORTHO II show in both images the measurements obtained with the device according to the state of the art. FIG. 7b then shows the same curve as FIG. 7a, but with reduced sensitivity. The markers “|” indicates the beginning of the nerve's response to stimulation, the markers A− and A+ indicate its amplitude range.

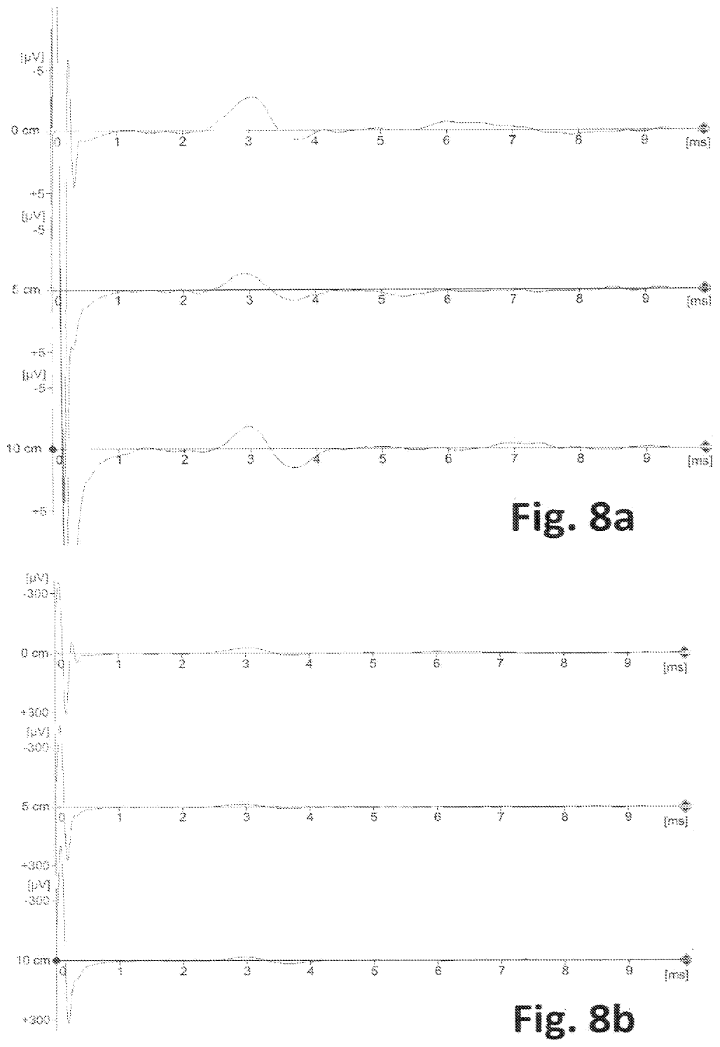

FIGS. 8a and 8b represent the dependence of the size of the stimulation artifact measured during stimulation performed on the device described above for several distances between the connection point of the collection electrode and the stimulation point, while the distance between the stimulation electrode and the recording electrodes was in this case about 20 cm. It is clear from FIGS. 8a and 8b that due to the influence of the method according to the invention, or the device according to the invention, the decay artifact is significantly reduced, so there is no problem in determining the beginning of the response. As a result of increasing the distance between the connection point of the collection electrode and the connection point of the stimulation electrode, the effect of removing the decay artifact of the stimulation artifact decreases, but as is clear, up to a distance of about Y of the distance between the stimulation electrode and the recording electrodes, the removal of this decay artifact of the stimulation artifact is reliable.

FIG. 6 then shows the result of the examination of somatosensory evoked potentials taken in three standardized places using the device from FIG. 3a. During this stimulation, the anode 4 and cathode 5 of the stimulation electrode of the electrical stimulator 1 were placed on the wrist of the patient, the collection electrode 6 was placed behind the wrist towards the body at a distance of about 3 cm, and the recording electrodes 8 were placed at Erb's point, in the area of the fifth cervical vertebra and on the scalp at standardized locations C3+ and C4+. As is clear from the graph, there was a substantial elimination of the stimulation artifact in all three scanned curves.

Mathematically, the reduction ratio of the stimulation effect can be determined as follows: To determine the amplitude of the stimulation artifact, it is possible to consider the model according to FIG. 3b. It is a model based on FIG. 3a supplemented with the impedance of the ZG collecting electrode 6. The basic assumption, which is very well fulfilled, is that the magnitude of the impedance of the Z_{G} collecting electrode 6 will be significantly lower than the magnitude of the impedances of the parasitic capacitances C_{S-P}, C_{S-P}, C_{S-W}, C_{S-U}, C_{S-B}, in the entire range of measured frequencies, that is

$$\vert ZG \vert << \vert ZS - P \vert , \vert ZG \vert << \vert ZW - W \vert , \vert ZG \vert << \vert ZS - W \vert etc .$$

whereas:

Z_{G} is the impedance between the collection electrode and the patient's body

Z_{S-P} is the capacitance impedance of C_{S-P}

Z_{W-W} is the capacitance impedance of C_{W-W}

Z_{S-W} is the capacitance impedance of C_{S-W}

ZS_{-U} is the capacitance impedance of C_{S-U}

Z_{S-B} is the capacitance impedance of C_{S-B}

It is clear to a man skilled in the art that the impedances mentioned above are actually transition impedances.

It is then possible to express the amplitude of the disturbing voltage causing the stimulation artifact as: U′_{ARTEF}=Z_{G}*I_{STIM}*(Z_{A}−Z_{C})/Z_{S-W}

When the original voltage value was already estimated U_{ARTEF}=I_{STIM}*(Z_{A}−Z_{C})/2,

then the ratio of reduction of disturbing voltages and thus of stimulation artifacts can be expressed as the ratio of U_{ARTEF} and U′_{ARTEF} values, while

$${U}_{ARTEF} / {U}_{ARTEF}^{′} = 1 / 2 \times {Z}_{S - W} / {Z}_{G}$$

The results of the measurements roughly correspond to the calculations made according to this model:

The electrical capacity of an individual conductor of a shielded cable with respect to its shield is around 150 pF for a 1.5 m long conductor, the size of the transition impedance of the connection of the collecting electrode with a large area is then around 2 kΩ, which is a realistically achieved value at normal stimulation current intensities. At a frequency of 10 kHz, which is the typical upper limit of the measured band, the suppression ratio reaches a value of approx. 12. As the frequency decreases, it continues to increase, because the capacitive component of the collection electrode is not as high with respect to its total impedance as with the impedance of the shield with respect to the connected centers of the connection cable, which is almost purely capacitive in nature.

### Verifying the Functionality of the Solution

The main evidence for the correctness of the indirect stimulation artifact suppression theory is shown in Figures Sa and Sb, which show the stimulation artifact. To improve the signal-to-noise ratio, the results of ten identical stimulations were averaged on the second and third graphs. Also for the sake of better visualization, the sensitivity was increased from 300 μV (microvolt) per division to 100 μV (microvolt) per division on both lower graphs.

The top graph of FIGS. 5a and 5b shows the course of the stimulation artifact. Both courses are almost identical, because at a small distance from the stimulation site, the directly propagated stimulation artifact clearly prevails.

In FIG. 5a, in the graph marked 10 cm, it can be seen that here already the indirect stimulation artifact propagated by capacitive coupling prevails over the directly propagated artifact via the galvanic path.

In FIG. 5a, in the graph marked 20 cm, only an indirectly propagated stimulation artifact is visible.

In FIG. 5b, in the graph marked 10 cm, only the remains of the directly propagated stimulation artefact are visible, the artefact indirectly propagated by capacitive couplings is almost completely led by the collection electrode back into the shielding of the electrical stimulator.

In FIG. 5b, in the graph marked 20 cm, only very small remnants of the indirectly propagated stimulation artefact, which failed to pass through the collection electrode, can be seen.

Evidence from practice is a comparison of the examination of the sensitive conduction of the median nerve using the orthodromic technique, while the results of stimulation with an electric stimulator according to the current state of the art and stimulation with an electric stimulator according to the invention to suppress the stimulation artifact were compared. When verifying the functionality of the invention with the orthodromic stimulation technique, the stimulation electrode was placed proximal to the wrist, while the recording electrodes were placed between the joints of the index finger. In both cases, the collection electrode was placed between the anode and the cathode of the stimulation electrode. In this case, the registration part was equipped with a grounding electrode, which was placed near the registration electrodes.

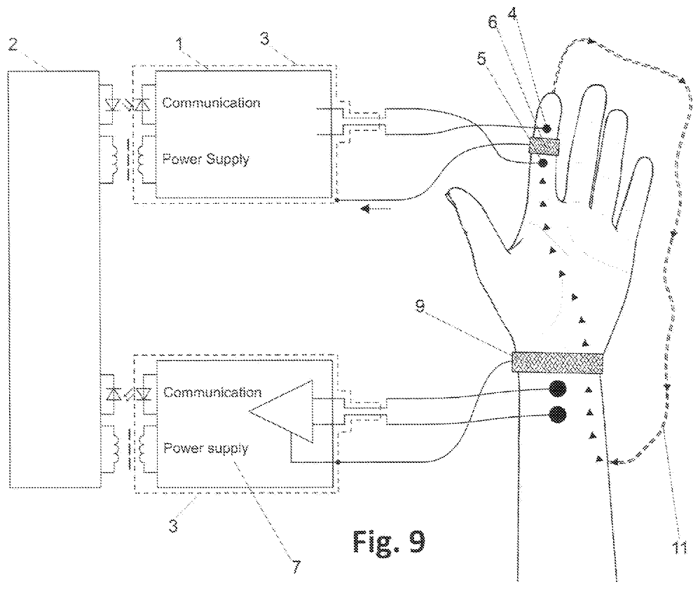

FIG. 9 shows the device according to the invention connected to the patient. This device includes an electric stimulator 1, a control unit 2, a stimulation electrode with an anode 4 and a cathode 5, a registration part 7 with registration electrodes 8 and a ground electrode 9. The electric stimulator 1 is equipped with an electric shield 3, which is connected to a collection electrode 6. Further in FIG. 9, the pertuberation touch line 11 shows a connection between a patient's finger and a place on the patient's body, which is behind the collecting electrode 6, in this case even behind the recording electrodes 8. The used electrical stimulator 1 was a single-channel electrical current stimulator of the TruTrace electromyograph manufactured by Deymed, placed in a plastic cover. Registration part 7 was created by one channel of a standard four-channel box of EMG amplifiers of the same electromyograph. The low capacitive coupling was ensured by the fact that the electrical stimulator 1 and the box of EMG amplifiers of the registration part 7 were supplied via isolation transformers with a very low capacitive coupling between the windings, in the order of picofarad units. The electrical shielding 3 of the electrical stimulator 1 was made by a metal cover of this electrical stimulator 1, which has an electrical resistance in the order of tens of Ohms per square. This electrical shielding 3 was externally led out, being provided for enabling the connection of the collecting electrode 6. For the electrical stimulation, a shielded bipolar stimulating electrode with anode 4 and cathode 5 with a length of connection cable of 2 m and a spacing between the centers of anode 4 and cathode 5 was chosen, which were in the form of wetted felt with a diameter of 23 mm. The shielding of this stimulation electrode was electrically connected to the electrical shielding 3 of the electrical stimulator 1 and connected to the patient in the immediate vicinity of the stimulation site by means of a collection electrode 6, whereas the collection electrode 6 was placed between the cathode 5 and the anode 4. For the collection electrode 6, a standard tape ground was used electrode with a connecting cable length of 2 m and a tape width of approx. 1.5 cm. A shielded pair of adhesive pre-gelled recording electrodes widely used for recording electrophysiological responses was chosen for recording, connected by a 2 m shielded cable to one channel in the recording part 7 of the device, which here was the amplifier box. All consumable supplies were represented by probably the most commonly used standard supplies used routinely in most well-equipped clinical European workplaces.

As mentioned, the collection electrode 6 has to be placed on a patient at a distance corresponding to at most half of the distance between the stimulation electrode and the registration electrodes 8, preferably at most to a distance corresponding to a quarter of this distance, and most preferably the collection electrode is placed as close as possible to the stimulation site, in particular to a distance corresponding to 4 times the distance between anode 4 and cathode 5 of the stimulation electrode. This ensures that the distance to which a perturbation impulse can spread from the stimulation electrode of the electrical stimulator 1 via capacitive coupling, is as small as possible. It is particularly advantageous to place the collection electrode 6 directly at the stimulation site, for example by incorporating the collection electrode into the stimulation electrode. The dependence of the distance of the location of the collection electrode from the place in which the stimulation electrode is applied is documented in FIGS. 8a and 8b. The distance between the stimulation electrode and the recording electrode was 40 cm. The distance between the anode and the cathode of the used stimulation electrode was 23 mm. The first track represents the situation when the collection electrode was placed directly at the stimulation site, i.e. at a distance of 0 cm. On the second track, the collection electrode is moved to a distance of 10 cm from the stimulation electrode towards the registration site, and on the third track, the collection electrode is moved to a distance of 20 cm from the stimulation electrode towards the registration site. There is a gradual increase in the indirect stimulation artifact, visible in the initial part of the recording, i.e. in the first millisecond after stimulation. The increase of the indirect stimulation artifact is caused by the influence of the increasing electrical capacity of the increasing volume of the peninsula of the patient's body to the environment and thus also to the recording electrodes.

It is clear to a man skilled in the art that for proper functioning of the stimulation methods, proper connection of all electrodes, including the collection electrode, is required to minimize the transition impedance between the electrode and the tissue. The reduction of transient impedance is ensured using commonly known methods, especially using electroconductive gels, etc.

### Closing Summary

The proposed solution for suppressing the stimulation artifact has the greatest benefit in methods that are characterized by a low amplitude of the useful electrophysiological signal and the registration point is not located in the immediate vicinity of the stimulation site, i.e. most often in the somatosensory evoked potentials (SSEP) methods and in the measurement of the sensitive neurogram, especially when using the orthodromic technique. That is, in methods that are of great clinical importance and that are quite often interfered with, especially by an indirect stimulation artifact, which makes their evaluation difficult or requires the use of software corrections. However, in case of software corrections it is not guaranteed to restore the electrophysiological signal into its original form. The proposed solution can also be used in methods where it is useful to minimize the amplitude of direct and indirect stimulation artifacts during continuous recording, for example when using electrical stimulation in electrocorticography.

The mutual combination of a shielded electrical stimulator with a connected collection electrode near the stimulation site and the use of a shielded amplifier box is particularly advantageous if their low mutual capacitive coupling is ensured. However, the end of the limb must not come into contact with the patient's body, because a balancing current would begin to flow through the limb and prevent the proper functioning of the proposed solution. If this rule is known, it is easy to ensure its observance in practice, see FIG. 10.

It is particularly advantageous to use the proposed solution when the indirect stimulation artefact prevails, that is, when the registration point is not in the immediate vicinity of the stimulation site, because the proposed solution makes it possible to suppress the indirectly propagated stimulation artefact.

It is clear to a man skilled in the art that the embodiments and examples of implementation of the device and the method according to the invention, especially as regards the described embodiment of the device according to the invention, in particular the electrical stimulator itself and the electrodes and other parts of the device, are only illustrative and show possibly particularly advantageous embodiments of the invention. The above mentioned embodiments are not intended to be limiting the invention to these particular embodiments. The subject of the invention is defined only by the appended patent claims.

The proposed solution of the method of suppressing the stimulation artifact during electrical stimulation as well as the device for suppressing the stimulation artifact has the greatest benefit for such neurophysiological methods that are characterized by a low amplitude of the useful electrophysiological signal and the place of registration is not in the immediate vicinity of the stimulation site, i.e. in methods where the stimulation artifact spreads from the point of stimulation to the point of registration mostly indirectly. The method according to the invention is particularly useful for somatosensory evoked potentials (SSEP) methods and for measuring the sensitive neurogram, especially when using the orthodromic technique. These procedures are most often used in electromyography and intraoperative monitoring, possibly also in electrocorticography using electrical stimulation. The invention is particularly advantageous for use in methods that are quite often disturbed by an indirect stimulation artifact, which makes it difficult to evaluate the registered signal, or often require the use of software corrections. However, such additional corrections are not always guaranteed to restore the electrophysiological signal to its original form.

### LIST OF REFERENCE NUMERALS

1 electrical stimulator
2 control unit
3 shielding
4 anode stimulation electrodes
5 cathode stimulation electrode
6 collection electrode
7 registration part
8 registration electrodes
9 grounding electrode
10 isolated peninsula
11 perturbation touch line

1. A method of reducing the stimulation artifact when performing electrical stimulation, in which a stimulation pulse is generated by an electrical stimulator connected to a stimulation electrode in a device for performing electrical stimulation, where this stimulation pulse is applied through a stimulation electrode placed on 2 patient and through registration electrodes placed on the patient at a distance from the stimulation electrode, the response to this stimulation impulse is measured, comprising
a) creating a closed equipotential surface around the electrical stimulator by complete electrical shielding of the electrical stimulator from the rest of the electrical stimulator and from its surroundings;
b) ensuring a low capacitive coupling between the electrical stimulator itself and the remaining parts of the electrical stimulation device;
c) connecting the shielding of the stimulation electrode to the closed equipotential surface of the electrical stimulator:
d) connecting, to the closed equipotential surface of the electrical stimulator and/or to the shielding of the stimulation electrode, a collection electrode designed to be placed on the patient, while this collection electrode is placed on the patient between the stimulation electrode and the registration electrodes, at a distance corresponding at most to one-half of the distance between the registration electrodes and the stimulation electrode.

2. The method according to claim 1, wherein, during stimulation, no current loop on the patient bypassing the connection point of the collection electrode can be created.

3. The method according to claim 1, wherein the collection electrode is placed as close as possible to the location of the stimulation electrode.

4. The method according to claim 1, wherein the collection electrode is placed between the individual poles of the stimulation electrode.

5. A device for electrical stimulation reducing the stimulation artifact, comprising:
an electrical stimulator (1) designed to be connected to a stimulation electrode,
a stimulation electrode including an anode (4) and a cathode (5),
recording electrodes (8) including an active electrode and a reference electrode,
a recording part (7) for connection with the recording electrodes, and
a control unit made at least for controlling the electric stimulator,
wherein the electric stimulator (1) is equipped with an electric shield (3) of the electric stimulator to create an equipotential conductive surface surrounding this electric stimulator (1), wherein the shielding is provided with a connection connector for connection to the shielding of the stimulation electrode or is directly connected to this shielding, and wherein the shielding (3) of the electrical stimulator (1) and/or to the shielding of the stimulation electrode is provided for connection to the collecting electrode (6) or is connected to the collecting electrode (6). and wherein at least between the electrical stimulator (1) and the remaining parts of the device, a low capacitive coupling is provided.

6. The device according to claim 5, wherein the electrical stimulator (1) is arranged in a cover made of electrically conductive material and/or in a cover made of electrically non-conductive material provided with an overall adhesive foil of electrically conductive foil and/or in a cover made of electrically non-conductive material provided with an electrically conductive varnish coating.

7. The device according to claim 6, wherein the electrically conductive film foil and/or electrically conductive varnish coating and/or metal cover have a resistance of at most 100 Ohm per square.

8. The device according to claim wherein the collection electrode (6) is a part of the stimulation electrode, being directly connected to the shielding of the stimulation electrode.

9. The device according to claim 8, wherein the collection electrode (6) is located between the anode (4) and the cathode (5) of the stimulation electrode.

10. The device according to claim 6, wherein the electrically conductive film foil and/or electrically conductive varnish coating and/or metal cover have a resistance of at most no more than 10 Ohm per square.

11. The device according to claim 6, wherein the electrically conductive film foil and/or electrically conductive varnish coating and/or metal cover have a resistance of at most no more than 1 Ohm per square.
